(19) United States
(12) Patent Application Publication
(10) Pub. No.: US 2012/0039860 A1
(43) Pub. Date: Feb. 16, 2012

(54) COMPOSITIONS AND METHODS FOR IMPROVED ORAL HEALTH

(75) Inventors: Zamzam Kabiry Roughead, Plymouth, MN (US); Kala Marie Kaspar, Vaud (CH)

(73) Assignee: NESTEC S.A., Vevey (CH)

(21) Appl. No.: 13/139,787

(22) PCT Filed: Dec. 14, 2009
(86) PCT No.: PCT/US09/67809

§ 371 Date: Jul. 8, 2011

Related U.S. Application Data
(60) Provisional application No. 61/123,903, filed on Apr. 10, 2008.
(60) Provisional application No. 61/240,829, filed on Sep. 9, 2009.

(51) Int. Cl. A61K 35/74; A61P 1/12; A61P 31/10; A61P 11/00; C12N 1/20; A61P 31/04

(52) U.S. Cl. 424/93.44; 435/253.4; 435/252.9; 435/252.1; 424/93.45; 424/93.4

(57) ABSTRACT

Compositions and methods for preventing and treating illnesses associated with oral health are provided. In a general embodiment, the compositions and methods of the present disclosure can improve oral health by reducing the incidence of aspiration pneumonia, community-acquired pneumonia and nosocomial pneumonia, allowing for less gingivitis and plaque, and providing improved tongue flora. By utilizing probiotics naturally present in the oral cavity or gastrointestinal tract, the compositions and methods in embodiments of the present disclosure can reduce anaerobic gram-negative bacilli and increase the presence of the normal flora. By preventing and treating problems associated with oral health in a patient, subsequent health problems can be prrevented or mitigated, which can result in reduced health care costs for the patient in the future.

### BACKGROUND

The present disclosure is direct to medical treatments. More specifically, the present disclosure is directed to compositions and methods for improving the oral health of an individual.

Epidemiological studies estimate a prevalence rate for dysphagia among those over the age of 5 years to be 16% to 22% among individuals. There are 2 broad categories of dysphagia: (i) esophageal dysphagia, and (ii) oral pharyngeal dysphagia.

Esophageal dysphagia affects a large number of individuals of all ages, but is generally treatable with medications and is considered a less serious form of dysphagia. Esophageal dysphagia is often a consequence of mucosal, mediastinal, or neuromuscular diseases. Mucosal (intrinsic) diseases narrow the lumen through inflammation, fibrosis, or neoplasia associated with various conditions (peptic stricture secondary to gastroesophageal reflux disease, esophageal rings and webs [sideropenic dysphagia or Plummer-Vinson syndrome], esophageal tumors, chemical injury [e.g., caustic ingestion, pill esophagitis, sclerotherapy for varices], radiation injury, infectious esophagitis, and eosinophilic esophagitis). Mediastinal (extrinsic) diseases obstruct the esophagus by direct invasion or through lymph node enlargement associated with various conditions (tumors [e.g., lung cancer, lymphoma], infections [e.g., tuberculosis, histoplasmosis], and cardiovascular [dilated auricula and vascular compression]). Neuromuscular diseases may affect the esophageal smooth muscle and its innervation, disrupting peristalsis or lower esophageal sphincter relaxation, or both, commonly associated with various conditions (achalasia [both idiopathic and associated with Chagas disease], scleroderma, other motility disorders, and a consequence of surgery [i.e., after fundoplication and antireflux interventions]). It is also common for individuals with intraluminal foreign bodies to experience acute esophageal dysphagia.

Oral pharyngeal dysphagia, on the other hand, is a very serious condition and is generally not treatable with medication. Oral pharyngeal dysphagia also affects individuals of all ages, but is more prevalent in older individuals. Worldwide, oral pharyngeal dysphagia affects approximately 22 million people over the age of 50. Oral pharyngeal dysphagia is often a consequence of an acute event, such as a stroke, brain injury, or surgery for oral or throat cancer. In addition, radiotherapy and chemotherapy may weaken the muscles and degrade the nerves associated with the physiology and nervous innervation of the swallow reflex.

It is also common for individuals with progressive neuromuscular diseases, such as Parkinson's Disease, to experience increasing difficulty in swallowing initiation. Representative causes of oropharyngeal dysphagia include those associated neurological illnesses (brainstem tumors, head trauma, stroke, cerebral palsy, Guillain-Barre syndrome, Huntington's disease, multiple sclerosis, polio, post-polio syndrome, Tardive dyskinesia, metabolic encephalopathies, amyotrophic lateral sclerosis, Parkinson's disease, dementia), infectious illnesses (diphtheria, botulism, Lyme disease, syphilis, mucositis [herpetic, cytomegalovirus, candida, etc.], autoimmune illnesses (lupus, scleroderma, Sjogren's syndrome), metabolic illnesses (amyloidosis, cushing's syndrome, thyrotoxicosis, Wilson's disease), myopathic illnesses (connective tissue disease, dermatomyositis, myasthenia gravis, myotonic dystrophy, oculopharyngeal dystrophy, polymyositis, sarcoidosis, paraneoplastic syndromes, inflammatory myopathy), iatrogenic illnesses (medication side effects [e.g., chemotherapy, neuroleptics, etc.], post surgical muscular or neurogenic, radiation therapy, corrosive [pill injury, intentional]), and structural illnesses (cricopharyngeal bar, Zenker's diverticulum, cervical webs, oropharyngeal tumors, osteophytes and skeletal abnormalities, congenital [cleft palate, diverticulae, pouches, etc.]).

Aspiration pneumonia is a common clinical consequence of dysphagia. The condition often requires acute hospitalization and emergency room visits. Among those that develop pneumonia due to aspiration, the differential diagnosis of “aspiration pneumonia” is not necessarily indicated as a result of current care practices.

Based on US healthcare utilization surveys from recent years, pneumonia accounted for over 1,000,000 hospital discharges and an additional 392,000 were attributable to aspiration pneumonia. Individuals who have general pneumonia as the principal diagnosis have a mean 6 day hospital length of stay and incur over $18,000 in costs for hospital care. It is expected that aspiration pneumonia would carry higher costs for hospital care based on a mean 8 day length of hospital stay.

Pneumonia is life threatening among persons with dysphagia, and the odds of death within 3 months is ˜50%. In addition, an acute insult such as pneumonia often initiates the downward spiral in health among elderly. An insult is associated with poor intakes and inactivity, resulting in malnutrition, functional decline, and frailty. For example, elderly commonly aspirate oropharyngeal contents during sleep.

The risk of aspiration pneumonia is greatest when periodontal disease, dental caries and poor oral hygiene are compounded by swallowing disease, feeding problems and poor functional status. Aspiration pneumonia is thought to be caused by the aspiration of colonized nasopharynx or oropharynx material secondary to dysphagia. Forceful coughing, active ciliary transport and normal immune response are presumed to be protective but are inadequate. Microflora present in the oral cavity because of poor oral hygiene have been associated with aspiration pneumonia.

The current standard of care for reducing incidence of aspiration pneumonia in patients with clinically-diagnosed swallowing disorders is chemical disinfection and use of antimicrobial agents to improve oral health. Additionally, another method called selective decontamination is used. This method is a prophylactic technique in which antimicrobials eradicate aerobic gram negative bacteria from the oropharynx while preserving the normal oral microbial flora. The agents include oral antimicrobial gels with antibiotics, liquid suspensions with same administered through a nasogastric (“NG”) tube, intravenous (“IV”) antibiotics and stringent infection control. While this method is superior to use of broad spectrum antibiotics that indiscriminately eradicate all bacteria (beneficial and harmful), it does not re-inoculate the oral cavity with the beneficial bacterial thus the patient does not re-establish balanced microflora and remains susceptible to onset of oral infections such as C. albicans (thrush).

The use of selective decontamination is also limited due to the risk of bacterial developing resistance to the antibiotics. In addition, the efficacy of this procedure in reducing aspiration pneumonia is questionable as in a recent meta-analysis, it was concluded that oral decontamination with chlorhexidine (“CHX”) could prevent ventilator associated pneumonia, but the strategy does not reduce the time on ventilator, the length of stay in the intensive care unit (“ICU”) or rates of mortality.

### SUMMARY

The present disclosure relates to compositions and methods for preventing and treating illnesses associated with oral health. In a general embodiment, the compositions and methods of the present disclosure can provide: (i) a reduction in the incidence of aspiration pneumonia, (ii) less gingivitis and plaque, and (iii) improved tongue flora. The source of chronic microbial challenge to the host can be targeted, which can reduce chronic inflammation on the host, help to restore an adequate immune response to physiological challenges and reduce an individual's risk of infections. By preventing and treating problems associated with oral health in a patient, subsequent health problems can be prevented or mitigated, which can result in reduced health care costs for the patient in the future.

In a general embodiment, the present disclosure provides a nutritional composition for improving oral health. The composition includes a therapeutically effective amount of a beneficial bacteria such as Lactobacillus reuteri, Lactobacillus plantarum, streptococcus Salivarius, Streptococcus salivarius K12, Lactobacillus reuteri ATCC55730, Lactobacillus johnsonii Lal, Lactobacillus plantarum 299v, Lactobacillus rhamnosus GG Streptococcus thermophilus NCC 1561, Lactococcus lactis NCC2211 (Pelargon strain), and Lacteol, and Other Ingredients with desired properties in accordance with the present invention include: CGMP which has been shown in testing to prevent binding of pathogens or a combination thereof. The beneficial bacteria can include one or more bacteria normally indigenous to the oral cavity. For example, the beneficial bacteria can include one or more strains normally indigenous to the oral cavity such as streptococcus Salivarius K12, Lactobacillus platarum 299, Lactobacillus platarum 299v or a combination thereof. The beneficial bacteria can be living or inactivated.

In an embodiment, the composition is in a form such as liquids, solids, semisolids or a combination thereof. The composition can be a complete oral nutritional supplement. The composition can also be in a form such as lozenges, lollipops, sachets, dissolvable films or a combination thereof

In an embodiment, the composition is in a form such as a food, a beverage and combinations thereof. The composition can include an ingredient such as a thickener. In an alternative embodiment, the composition is a topical compound that can be applied to the surface of the oral cavity.

In another embodiment, the present disclosure provides a method of reducing the incidence of aspiration pneumonia. The method comprises administering to a patient at risk of or having aspiration pneumonia a therapeutically effective amount of a composition comprising a beneficial bacteria selected from the group consisting of Lactobacillus reuteri, Lactobacillus plantarum, streptococcus Salivarius, Streptococcus salivarius K12, Lactobacillus reuteri ATCC55730, Lactobacillus johnsonii Lal, Lactobacillus plantarum 299v, Lactobacillus rhamnosus GG Streptococcus thermophilus NCC 1561, Lactococcus lactis NCC2211 (Pelargon strain), and Lacteol, and Other Ingredients with desired properties in accordance with the present invention include: CGMP which has been shown in testing to prevent binding of pathogens and combinations thereof

In an alternative embodiment, the present disclosure provides a method of improving oral health. The method comprises administering to a patient at risk of or having oral health problems a composition comprising a therapeutically effective amount of a beneficial bacteria selected from the group consisting of Lactobacillus reuteri, Lactobacillus plantarum, streptococcus Salivarius, Streptococcus salivarius K12, Lactobacillus reuteri ATCC55730, Lactobacillus johnsonii Lal, Lactobacillus plantarum 299v, Lactobacillus rhamnosus GG Streptococcus thermophilus NCC 1561, Lactococcus lactis NCC2211 (Pelargon strain), and Lacteol, and Other Ingredients with desired properties in accordance with the present invention include: CGMP which has been shown in testing to prevent binding of pathogens and combinations thereof.

In yet another embodiment, the present disclosure provides a method of reducing healthcare costs. The method comprises administering to a patient at risk of or having oral health problems a composition comprising a therapeutically effective amount of a beneficial bacteria selected from the group consisting of Lactobacillus reuteri, Lactobacillus plantarum, streptococcus Salivarius, Streptococcus salivarius K12, Lactobacillus reuteri ATCC55730, Lactobacillus johnsonii Lal, Lactobacillus plantarum 299v, Lactobacillus rhamnosus GG Streptococcus thermophilus NCC 1561, Lactococcus lactis NCC2211 (Pelargon strain), and Lacteol, and Other Ingredients with desired properties in accordance with the present invention include: CGMP which has been shown in testing to prevent binding of pathogens. and combinations thereof. The reduction in healthcare costs can be due to decreased incidences of aspiration pneumonia or general pneumonia that may not be differentially diagnosed. Alternatively, the reduction in healthcare costs can be due to a reduced treatment of secondary infections and/or prevention of a downward spiral in health. This can include, for example, loss of functionality, frailty, disability and death.

The reduction in healthcare costs be due to improved overall health of the patient or due to decreased dental costs. The reduction in healthcare costs can be due to decreased utilization of hospitals and skilled nursing facilities. Decreased utilization can be fewer days, fewer number of admissions, fewer ER visits, decreased incidences of aspiration pneumonia. The reduction in healthcare costs can also be due to decreased utilization of specialized care.

The reduction in healthcare costs can also be due to decreased utilization of antibiotics and/or artificial ventilation and/or pulmonary rehabilitation and/or physical therapy post-ventilation and/or intravenous fluids. The decreased utilization can be due to a decreased need for prevention of aspiration pneumonia. The decreased utilization can be due to treatment of sequellae from antibiotic use. The sequellae can be fungal infections (thrush), or urinary tract infections (“UTIs”), or C. difficile associated diarrhea or a combination thereof. The sequellae can also be infections from antibiotic resistant bacteria, methicillin-resistant Staphylococcus aureus, or a combination thereof. The decreased sequellae from antibiotic use can be due to decreased incidences of infections from antibiotic resistant bacteria.

An advantage of the present disclosure is to provide a composition for improving oral health.

Another advantage of the present disclosure is to provide a method for improving oral health.

Yet another advantage of the present disclosure is to provide a method for reducing the incidence of aspiration pneumonia.

Still another advantage of the present disclosure is to provide a method of reducing health care costs.

Additional features and advantages are described herein, and will be apparent from the following Detailed Description.

### DETAILED DESCRIPTION

The present disclosure relates to compositions and methods for preventing and treating illnesses associated with oral health. In alternative embodiments, the compositions and methods can be used to reduce improve oral health such reducing the incidence of aspiration pneumonia. The compositions and methods can also be used to reduce the healthcare costs associated with treating the effects of adverse oral health conditions.

It has been surprisingly found that using a therapeutically effective amount of bacteria or probiotics naturally present in the oral cavity can reduce anaerobic gram-negative bacilli (“AGNB”) and increase the presence of the normal flora thereby re-introducing the good bacterial to the mouth without increasing the risk of antibiotic resistance or antibiotic-associated adverse outcomes. Additionally, side effects such as discoloration of the teeth, irritation of mucosa or even serious allergic reactions can be mitigated or eliminated. As the selected probiotics will have beneficial systemic effects, patients can benefit not only from the localized improvement of oral health (with important end benefit of reduced aspiration pneumonia), but also improved general immunity.

The compositions and methods in embodiments of the present disclosure are beneficial for individuals or patients with clinical (diagnosed), subclinical (undiagnosed), or at risk of disorders of swallowing. Patients with swallowing difficulties such as those clinically diagnosed with dysphagia or those at risk of developing dysphagia can be malnourished or elderly and have Parkinson's, Alzheimer disease, dementia, head and neck cancer, stroke, Down's syndrome or conditions leading to protruded tongue.

The compositions and methods can provide health benefits to patients at risk or having silent aspiration (e.g. aspirate during sleep or any other time such as while reclined) and those at risk of or who have had pneumonia (e.g. recurrent pneumonias, immune-compromised persons). In addition, the compositions and methods can provide health benefits to those who are at risk of or have poor oral health (e.g. secondary to medication use, decreased saliva production, smoking/tobacco use, alcohol use, liver failure, infrequently practice oral hygiene methods, functionally-impaired who require assistance with oral care).

As used herein the term “patient” is preferably understood to include an animal, especially a mammal, and more especially a human that is receiving or intended to receive treatment, as it is herein defined.

As used herein, “mammal” includes but is not limited to rodents, aquatic mammals, domestic animals such as dogs and cats, farm animals such as sheep, pigs, cows and horses, and humans. Wherein the term mammal is used, it is contemplated that it also applies to other non-mammal animals that are capable of the effect exhibited or intended to be exhibited by the mammal.

As used herein, “complete nutrition” are preferably nutritional products that contain sufficient types and levels of macronutrients (protein, fats and carbohydrates) and micronutrients to be sufficient to be a sole source of nutrition for the animal to which it is being administered to.

As used herein, “effective amount” is preferably an amount that prevents a deficiency, treats a disease or medical condition in an individual or, more generally, reduces symptoms, manages progression of the diseases or provides a nutritional, physiological, or medical benefit to the individual. A treatment can be patient- or doctor-related. In addition, while the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, “incomplete nutrition” are preferably nutritional products that do not contain sufficient levels of macronutrients (protein, fats and carbohydrates) or micronutrients to be sufficient to be a sole source of nutrition for the animal to which it is being administered to.

As used herein, “Long term administrations” are preferably continuous administrations for more than 6 weeks.

The term “microorganism” is meant to include the bacterium, yeast and/or fungi, a cell growth medium with the microorganism or a cell growth medium in which microorganism was cultivated.

As used herein, a “Prebiotic” is preferably a food substances that selectively promote the growth of beneficial bacteria or inhibit the growth of pathogenic bacteria in the intestines. They are not inactivated in the stomach and/or upper intestine or absorbed in the GI tract of the person ingesting them, but they are fermented by the gastrointestinal microflora and/or by probiotics. Prebiotics are for example defined by Glenn R. Gibson and Marcel B. Roberfroid, Dietary Modulation of the Human Colonic Microbiota: Introducing the Concept of Prebiotics, J. Nutr. 1995 125: 1401-1412.

As used herein, Probiotics micro-organisms (hereinafter “probiotics”) are preferably microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. (Salminen S, Ouwehand A. Benno Y. et al “Probiotics: how should they be defined” Trends Food Sci. Technol. 1999:10 107-10). In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products.

As used herein, “Short term administrations” are preferably continuous administrations for less than 6 weeks.

The terms “Tolerable Upper Limit and Upper Limit (UL)” are preferably meant to include the maximum nutrient level that will likely pose no risk of adverse events.

As used herein, the terms “treatment”, “treat” and “to alleviate” is preferably to both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment”, “treat” and “to alleviate” are also intended to include the potentiation or otherwise enhancement of one or more primary prophylactic or therapeutic measure. The terms “treatment”, “treat” and “to alleviate” are further intended to include the dietary management of a disease or condition or the dietary management for prophylaxis or prevention a disease or condition

All dosage ranges contained within this application are intended to include all numbers, whole or fractions, contained within said range.

As used herein, “Comensal bacteria” are those microorganisms that help the digestion of food and acquiring of nutrients such as vitamins B and K, and assisting the immune system in preventing the colonization of pathogens that cause disease by competing with them.

As used herein, a “Comensal Effect” is when a microorganism, by itself, or aids another organism in helping the digestion of food and acquiring of nutrients such as vitamins B and K, and assisting the immune system in preventing the colonization of pathogens that cause disease by competing with them.

The compositions and methods in embodiments of the present disclosure provide superior alternatives to current standards of care for improving oral health. The compositions and methods can be used to supplement beneficial bacteria in the oropharynx of the patient. The compositions and methods can provide competitive inhibition through displacing pathogenic bacteria by competing for adhesion sites and nutrients. The compositions and methods can restore microfloral balance in the patient's oropharynx, esophagus, and the rest of the gastrointestinal (“GI”) tract.

The compositions and methods do not need to be used in conjunction with ventilators that can collect pathogens (e.g., reservoir) and provide a place to attack them wherein. Under normal circumstances, there is no place for the bacteria to be static/colonize, etc.

The compositions and methods can utilize natural, indigenous probiotics that reduce salivary pathogenic bacterial load in the oropharynx and nasopharynx. Through competitive inhibition, the natural, indigenous probiotics can offer oral health without killing the other bacteria or inducing antibiotic-resistance. However, certain probiotics can kill pathogenic bacteria and inhibit their reproduction (e.g. 1. reuteri produce natural antiobiotics that kill pathogens). The probiotics can compete with receptor signaling sites that mediate systemic inflammation. As a result, the ingested probiotics can then confer immune and gut health benefits. An advantage of this ingestion can be multifold as it addresses the immune response of the patient from the oral cavity to systemic immune response.

The compositions and methods in alternative embodiments of the present disclosure can reduce the use of antibiotics in a dysphagia patient, reduce pathogenic bacterial load in a dysphagia patient, reduce pathogenic bacterial load in the oropharynx of a dysphagia patient, reduce pathogenic bacterial load in the nasopharynx of a dysphagia patient, reduce pathogenic bacterial load in the oropharynx and nasopharynx of a dysphagia patient, reduce pathogenic bacterial load in dysphagia patients (e.g. wherein the pathogenic bacterial is AGNB), and improve the overall health and cardiovascular of the patient.

The compositions and methods in embodiments of the present disclosure can utilize a therapeutically effective amount of live or inactivated beneficial bacteria (e.g. probiotics) as follows:

| a. | Delivered orally | |
|---|---|---|
| | i) | Alone |

| | |
|---|---|
| (1) | Liquids |
| (2) | Semi-solids |
| (3) | Lozenges |

| | |
|---|---|
| (a) | Including lollipops |

| | |
|---|---|
| (4) | Sachets |
| (5) | Films |

| | |
|---|---|
| ii) | In conjunction with |

| | |
|---|---|
| (1) | Liquids |

| | |
|---|---|
| (a) | Oral nutritional supplement s |

| | |
|---|---|
| (i) | Premixed in |
| (ii) | Modules to be added |
| (iii) | Complete oral nutritional supplements (“ONS”) for use as directed |
| | by a medical professional - that contains protein, carbohydrate, fat |
| | and micronutrients (fiber optional) sufficient for sustaining life and |
| | other bioactive compounds such as phytochemicals, bacterial by |
| | products, nucleotides, etc |
| (iv) | Incomplete ONS missing one or more of the components of a |
| | complete ONS |
| (v) | Contains enriched levels of one or more of the components of a |
| | complete ONS |

| | |
|---|---|
| (b) | Other beverages |

| | |
|---|---|
| (i) | Juices, milk, soft drinks, coffee, teas, water |

| | |
|---|---|
| (2) | Solids |

| | |
|---|---|
| (i) | Modules to be sprinkled on food |

| | |
|---|---|
| 1. | Including gravy and sauces |
| 2. | Includes semi-solids |
| | a. Puddings, jello, jellies |

| | |
|---|---|
| (ii) | Bars |
| (iii) | Other topical compounds such as vitamin E, vitamin A, Zn, vitamin |
| | C, nucleotides and other bioactive compounds such as |
| | phytochemicals, bacterial by products, nucleotides, etc. |
| (iv) | Modified consistency food products/mixes (e.g., puree) |

| | |
|---|---|
| (3) | Other topical compounds (e.g., Vitamin C and Zinc) |
| (4) | Improve oral health and/or immunity |

| | |
|---|---|
| (i) | Substances that affect the binding capacity of pathogens with |
| | receptor signaling sites that mediates local and/or systemic |
| | inflammation |

| | |
|---|---|
| (b) | Decrease pathogenic load |

| | |
|---|---|
| (i) | Substance that create an unfavorable environment for the growth of |
| | pathogens |

| | |
|---|---|
| 1. | Replaces or binds the essential nutrients (e.g., fuel-sugar, free- |
| | water content) that support the growth of pathogens |
| 2. | Alters the pH |
| 3. | Generates an anti-microbial substance that inhibits pathogen |
| | growth |

| | |
|---|---|
| iii) | Thickeners |

| | |
|---|---|
| (1) | Starches |
| (2) | Gum-based |
| (3) | Other plant extracts |

| | |
|---|---|
| (a) | Bamboo shoot |

| | |
|---|---|
| (4) | Proteins |

| b. | Bacteria indigenous to the oral cavity | |
|---|---|---|
| | i) | streptococcus Salivarius |
| | ii) | Lactobacillus reuteri |
| | iii) | Lactobacillus platarum 299 or 299v |
| c. | Bacteria non-indigenous to the oral cavity | |
| d. | Prebiotics and nucleotides | |
| e. | Substances that promote non-pathogenic bacteria (i.e. like prebiotics in the | |
| | intestine) | |
| | i) | Optimizes the environment (e.g., pH, energy source availability) to favor |
| | | growth of non-pathogenic bacteria, availability of free-water |
| | ii) | Other Ingredients with desired properties in accordance with the present |
| | | invention include: CGMP which has been shown in testing to prevent binding |
| | | of pathogens and is known to be effective against Streptococcus mutans and |
| | | Streptococcus sobrinus. |

The repeated consumption of the compositions in embodiments of the present disclosure as part of daily life can improve oral health by replacing the pathogenic bacteria with beneficial, non-pathogenic bacteria. Oral health can also be improved as a result of the releasing of beneficial chemicals by the bacteria (e.g. anti-microbials). In addition, as the individual aspirates their saliva, the pathogenic bacterial load will be low and the likelihood of onset of aspiration pneumonia can be reduced.

Non-limiting examples of bacteria to be used in accordance with this invention include one or more of: Streptococcus salivarius K12, Lactobacillus reuteri ATCC55730, Lactobacillus johnsonii Lal, Lactobacillus plantarum 299v, Lactobacillus rhamnosus GG Streptococcus thermophilus NCC 1561, Lactococcus lactis NCC2211 (Pelargon strain), and Lacteol.

Streptococcus salivarius K12, which produces produce salivaricin A and B, in testing has been shown to be effective against Streptococus pyogenes, Micrococcus luteus, Streptococcus anginosis, Eubacterium saburreum, Micromonas micros, Moraxella, Prevotella intermedia, and Porphyomonas gingivalis,

Lactobacillus reuteri ATCC55730, which produces reuterin, in testing has been shown to be effective against: Streptococcus mutans, EHEC Escherichia coli, ETEC Escherichia coli, Salmonella enterica, Shigella sonnei, Vibrio cholerae. Additionally, Lactobacillus reuteri ATCC55730 has a commensal effect on L. casei ATCC 334, L. johnsonii ATCC33200, L. acidophilus ATCC 4356, L. gasseri ATCC 33323, Clostridium difficile, Eubacterium eligens, Bifidobacterium longum var infantis, Eubacterium biforme, Bifidobacterium longum, Bifidobacterium catenulatum, Bacteroides vulgatus, and Bacteroides thetaiotaomicron.

Lactobacillus johnsonii Lal, which produces H202, in testing has been shown to be effective against: Escherichia coli (ETEC, EPEC), Salmonella typhimurium, Yersinia pseudotuberculosis, Helocobacter pylori, Toxin A from Clostridium difficile, Shigella flexneri, Klebsiella pneumoniae, Pseudomonas aeruginosa, Enterobacter cloacae, Staphylococcus aureus, and Listeria monocytogenes.

Lactobacillus plantarum 299v has been shown to help prevent colonisation of pathogens, and aids in preventing ventilation associated pneumoniae (VAP). Testing has shown that LP299v is effective against: Streptococcus mutans, Streptococcus sobrinus, and Escherichia coli.

Lactobacillus rhamnosus GG has also been shown to help prevent colonisation of pathogens, and aids in preventing ventilation associated pneumoniae (VAP). Testing has shown that Lactobacillus rhamnosus GG is effective against Streptococcus mutans, Streptococcus sobrinus, and Escherichia coli.

Streptococcus thermophilus NCC 1561 , in testing has been shown to be effective against Actinomyces viscosus, and Strepotcoccus sobrinus.

Lactococcus lactis NCC2211 (Pelargon strain), in testing has been shown to be effective against Actinomyces viscosus and Strepotcoccus sobrinus. Examples of Non-Replicating Micro-organisms include

Lacteol which acts by a mechanismof inhibition of adhesion of pathogens (adheres to Caco 2 and HT29-MRX cells) and has been shown in testing to prevent adhering of Lysteria monocytogenes, ETEC Escherichia coli, EPEC Escherichia coli, Yersinia pseudotuberculosis, and Salmonella typhimurium. Additionally, Lacteol has been shown to have antimicrobial activity against: Staphylococcus aureus, Listeria monocytogenes, Bacillus cereus, Salmonella typhimurium, Shigella flexneri, Escherichia coli, Klebsiella pneumoniae, Pseudomonas aeruginosa, and Enterobacter spp.

In yet another embodiment, the present disclosure provides a method of reducing healthcare costs. The method comprises administering to a patient at risk of or having oral health problems a composition comprising a therapeutically effective amount of a beneficial bacteria selected from the group consisting of Lactobacillus reuteri, Lactobacillus plantarum, streptococcus Salivarius, Streptococcus salivarius K12, Lactobacillus reuteri ATCC55730, Lactobacillus johnsonii Lal, Lactobacillus plantarum 299v, Lactobacillus rhamnosus GG Streptococcus thermophilus NCC 1561, Lactococcus lactis NCC2211 (Pelargon strain), and Lacteol and combinations thereof. The reduction in healthcare costs can be due to decreased incidences of aspiration pneumonia or general pneumonia that may not be differentially diagnosed. Alternatively, the reduction in healthcare costs can be due to a reduced treatment of secondary infections and/or prevention of a downward spiral in health. This can include, for example, loss of functionality, frailty, disability and death.

More specifically, the health economic benefits can be as follows:
Reduce hospitalizations, rehospitalizations, sub-acute care, transitional care, home health care, outpatient care, physician office visits and follow-up care
   Reduce medical costs to the health care system for aspiration pneumonia and general pneumonia that may not be differentially diagnosed
Reduced necessary specialized care
   Reduce medical costs to the health care system for dehydration, use of artificial ventilation, emergency room visits, pulmonary rehabilitation and physical therapy post-artificial ventilation, nosocomial infections and recurrence (e.g., urinary tract infections, C. difficile associated diarrhea)
Reduced need for antibiotics
   Fewer superbugs floating around (antibiotic resistant bacteria)
      Methicillin-resistant Staphylococcus aureus (MRSA)
      C. difficile
Saved costs for better overall health
   Cardiovascular, diabetes, metabolic syndrome
   Avoid the downward spiral in health that can lead to loss of functionality, frailty, increased risk of illness injury & death, disability, increased (formal & informal) caregiver burden, and institutionalization
Reduced costs of dental care or physician office visits

### EXAMPLES

By way of example and not limitation, the following examples are illustrative of various embodiments of the present disclosure.

### Example 1

Table 1 lists components for a complete feeding product (powder or liquid) appropriate for nutritional supplementation of patients with or at risk of dysphagia, those at high risk of aspiration, at high risk of pneumonia, and at risk of poor oral health.

TABLE 1

| Ingredient | Amount | Function |
|---|---|---|
| Probiotic Strain | >10,000 CFU (powder or | Bacteriotherapy |
| | in straw, cap or another | (displacement of |
| | delivery mechanism) | pathogenic bacteria) |
| Casemate | 75 g (20% of energy) | High quality protein |
| Canola oil | 50 g (30% of energy) | Fatty acid source and |
| | | energy |
| Maltodextrin | 188 g (50% of energy) | Carbohydrate and |
| | | energy source |
| Yeast extract | 2.5 g | RNA/nucleotide source |
| Vitamin premix | | |
| Mineral premix | | |
| Emulsifier | | |
| water | | |
| Optional ingredients | | As listed elsewhere |

### Example 2

Compositions having thickeners that are appropriate for nutritional supplementation of patients with or at risk of dysphagia, those at high risk of aspiration, at high risk of pneumonia, and at risk of poor oral health can include components of Table 2. The thickeners can be starches, gums or other plant or animal based thickeners.

TABLE 2

| Ingredient | Amount | Function |
|---|---|---|
| Probiotic Strain | >10,000 CFU (powder or | Bacteriotherapy |
| | in straw, cap or another | (displacement of |
| | delivery mechanism) | pathogenic bacteria) |
| Thickener | | Increases viscosity for |
| | | ease and safety of |
| | | swallowing |
| Optional nutrient: | | Enriches the diet in |
| (e.g., high quality | | essential or beneficial |
| protein) | | nutrients |

### Example 3

Modular powders or liquid supplements (e.g., protein) appropriate for nutritional supplementation of patients with or at risk of dysphagia, those at high risk of aspiration, at high risk of pneumonia, and at risk of poor oral health can include components of Table 3.

TABLE 3

| Ingredient | Amount | Function |
|---|---|---|
| Probiotic Strain | >10,000 CFU (powder or | Bacteriotherapy |
| | in straw, cap or another | (displacement of |
| | delivery mechanism) | pathogenic bacteria) |
| Nutrient (e.g., high | | Enriches the diet in |
| quality protein) | | essential or beneficial |
| | | nutrients |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A composition for improving health comprising a therapeutically effective amount of beneficial bacteria comprising at least one bacteria selected from the group consisting of:
streptococcus Salivarius;
Lactobacillus reuteri;
Lactobacillus platarum 299 or 299v;
Streptococcus salivarius K12;
Lactobacillus reuteri ATCC55730;
Lactobacillus johnsonii Lal;
Lactobacillus rhamnosus GG;
Streptococcus thermophilus NCC 1561;
Lactococcus lactis NCC2211 (Pelargon strain); and
Lacteol.

2. The composition as claimed in claim 1, wherein the beneficial bacteria is at least one bacteria normally indigenous to the oral cavity.

3. The composition as claimed in claim 1, wherein the composition comprises bacteria that are inactivated.

4. The composition as claimed in claim 1, wherein the composition is in a form selected from the group consisting of lozenge, lollipop, sachet and dissolvable film.

5. The composition as claimed in claim 1, wherein the composition comprises a topical compound.

6. The composition as claimed in claim 1, wherein the composition comprises a thickener.

7. A method for reducing the incidence of aspiration pneumonia comprising administering to a patient at risk of same a composition comprising a therapeutically effective amount of beneficial bacteria selected from the group consisting of: streptococcus Salivarius; Lactobacillus reuteri; Lactobacillus platarum 299 or 299v; Streptococcus salivarius K12; Lactobacillus reuteri ATCC55730; Lactobacillus johnsonii Lal; Lactobacillus rhamnosus GG; Streptococcus thermophilus NCC 1561; Lactococcus lactis NCC2211 (Pelargon strain); and Lacteol.

8. The method as claimed in claim 7, wherein the beneficial bacteria is at least one bacteria normally indigenous to the oral cavity.

9. The method as claimed in claim 7, wherein the composition comprises bacteria that are inactivated.

10. The method as claimed in claim 7, wherein the composition is in a form selected from the group consisting of lozenge, lollipop, sachet and dissolvable film.

11. The method as claimed in claim 7, wherein the composition comprises a topical compound.

12. The method as claimed in claim 7, wherein the composition comprises a thickener.

13. A method for improving oral health comprising administering to a patient a composition comprising a therapeutically effective amount of beneficial bacteria comprising at least one bacteria selected from the group consisting of: streptococcus Salivarius; Lactobacillus reuteri; Lactobacillus platarum 299 or 299v; Streptococcus salivarius K12, Lactobacillus reuteri ATCC55730; Lactobacillus johnsonii Lal; Lactobacillus rhamnosus GG; Streptococcus thermophilus NCC 1561; Lactococcus lactis NCC2211 (Pelargon strain); and Lacteol.

14. The method as claimed in claim 13, wherein the improved oral health leads to a reduction in the incidence of aspiration pneumonia.

15. The method as claimed in claim 13, wherein overall health of the patient is improved.

16. (canceled)

17. The method as claimed in claim 7, wherein pathogenic bacterial load in the oropharynx, or nasopharynx, or oropharynx and nasopharynx of a dysphagia patient is reduced.

18. The method as claimed in claim 7, wherein the risk of morbidity is reduced.

19. The method as claimed in claim 1 wherein healthcare costs are reduced, the reduction in healthcare costs being due to decreased: incidences of aspiration pneumonia, co-morbidities of aspiration pneumonia, treatment of secondary infections, treatment of downward spiral in health, treatment of sequellae from antibiotic use, and combinations thereof.

20. The method as claimed in claim 19 wherein the sequellae from antibiotic use is selected from the group consisting of fungal infections, urinary tract infections, c. difficile associated diarrhea, antibiotic resistant bacteria, methicillin-resistant Staphylococcus aureus, c. difficile, and combinations thereof.

21. The method as claimed in claim 19 wherein the reduction in healthcare costs are due to decreased utilization of at least one healthcare cost selected from the group consisting of: hospitals, skilled nursing facilities, emergency room, specialized care facilities, rehabilitation centers, antibiotics, artificial ventilation, pulmonary rehabilitation, physical therapy post-ventilation, intravenous fluids, dental services, and combinations thereof.

22. The method as claimed in claim 13 wherein healthcare costs are reduced, the reduction in healthcare costs being due to decreased: incidences of aspiration pneumonia, co-morbidities of aspiration pneumonia, treatment of secondary infections, treatment of downward spiral in health, treatment of sequellae from antibiotic use, and combinations thereof

23. The method as claimed in claim 22 wherein the sequellae from antibiotic use is selected from the group consisting of fungal infections, urinary tract infections, c. difficile associated diarrhea, antibiotic resistant bacteria, methicillin-resistant Staphylococcus aureus, c. difficile, and combinations thereof.

24. The method as claimed in claim 22 wherein the reduction in healthcare costs are due to decreased utilization of at least one healthcare cost selected from the group consisting of: hospitals, skilled nursing facilities, emergency room, specialized care facilities, rehabilitation centers, antibiotics, artificial ventilation, pulmonary rehabilitation, physical therapy post-ventilation, intravenous fluids, dental services, and combinations thereof.

25. The method as claimed in claim 13, wherein the beneficial bacteria is at least one bacteria normally indigenous to the oral cavity.

26. The method as claimed in claim 13, wherein the composition comprises bacteria that are inactivated.

27. The method as claimed in claim 13, wherein the composition is in a form selected from the group consisting of lozenge, lollipop, sachet and dissolvable film.

28. The method as claimed in claim 13, wherein the composition comprises a topical compound.

29. The method as claimed in claim 13, wherein the composition comprises a thickener.
